(19) United States
(12) Patent Application Publication
(10) Pub. No.: US 2019/0000822 A1
(43) Pub. Date: Jan. 3, 2019

(54) CHLORINE OR BROMINE SALTS OF CETYLPYRIDINIUM FOR USE IN THE TREATMENT OF CUTANEOUS AND ACUTE PORPHYRIAS AND PSORIASIS

(71) Applicant: Nicolae Arcasiu, Astoria, NY (US)

(72) Inventor: Nicolae Arcasiu, Astoria, NY (US)

(21) Appl. No.: 16/063,849

(22) PCT Filed: Feb. 24, 2016
(86) PCT No.: PCT/RO2016/000008

§ 371 Date: Jun. 19, 2018

(51) Int. Cl. A61K 31/4425; A61K 9/08; A61K 9/00; A61K 9/06; A61K 31/255; A61K 31/58; A61K 31/575; A61K 47/06; A61P 17/06; A61P 17/00

(52) U.S. Cl. CPC A61K 31/4425; A61K 9/08; A61K 9/0014; A61K 9/06; A61P 17/00; A61K 31/58; A61K 31/575; A61K 47/06; A61P 17/06; A61K 31/255

(57) ABSTRACT

The present invention relates to chlorine or bromine salts of cetylpyridinium for use in the treatment of porphyrias and psoriasis, as well as methods of treatment of these diseases in human patients by using hydro-alcoholic solutions of cetylpyridinium chloride or bromide.

The present invention relates to chlorine or bromine salts of cetylpyridinium for use in the treatment of porphyria and psoriasis, as well as to methods of treatment of these diseases in human patients using hydro-alcoholic solutions of cetylpyridinium chloride or bromide.

Uses of cetylpyridinium chloride and bromide are known as antiseptic within the composition of toothpastes, mouthwashes, nasal sprays, tablets or lozenges, being used to prevent and fight dental plaque and to treat mild infections of the oral cavity and throat (pharyngitis, laryngitis); inflammation of gums and mouth, symptoms such as stinging, dryness and irritation in throat; for hoarseness and bad breath. Furthermore, uses of cetylpyridinium chloride and bromide are known in the manufacture of pesticides.

Porphyrias are incurable genetic diseases with pathological manifestations due to heredity, by transmitting genetic information from parents to offspring. They are diseases caused by disorders in heme biosynthesis (non-protein fraction of hemoglobin) and characterized by the accumulation of precursors from the porphyrin synthesis in tissues. This leads to neurological or skin symptoms, sometimes both. Thus, porphyrias are classified, in terms of their symptoms, in acute porphyrias and cutaneous porphyrias. Acute porphyrias generally manifest with abdominal pain, vomiting, neuropathies and mental disorders. The onset of symptoms of acute porphyrias is related to triggers such as certain drugs (such as barbiturates, sulfonamides), certain viral infections. Cutaneous porphyrias manifest with skin abnormalities such as skin fragility, abrasions, rashes or painful skin blisters, and sometimes secondary infection, crusting, especially on the dorsal surface of the hands, neck and face. These cutaneous symptoms occur especially after sunlight exposure, by the action of the ultra-violet (UV) radiation as porphyrins react to these UV radiations. Healing is slow, with wounds and hypo- or hyper-pigmentations, atrophic scars, and sometimes calcified dystrophies. Currently, the treatment of porphyrias consists mainly of avoiding triggers: avoiding sunlight, UV exposure, alcohol consumption, certain drugs (e.g. barbiturates, sulfonamides), and vaccination against viral diseases, such as hepatitis. This means that patients will not be able to have a normal life, being forced to permanently avoid exposure to sunlight and/or other triggers. The administration of glucose or a drug chemically related to heme can be useful for the moment to treat attacks of acute porphyria, but with inconsistent results and high costs. In cutaneous porphyria phlebotomy (venous bleeding) is sometimes applied, consisting of drawing an amount of 250-350 ml of blood in the hospital every week for 4-5 weeks in a row, 2-3 times per year, which makes the treatment expensive, difficult and stressful for patients. It has also been tried bone marrow transplantation in children, but with uncertain outcomes and high costs. None of the above treatments cure the disease or vanish the symptoms. There are 8 types of porphyrins, and porphyrias are severe incurable genetic diseases.

Psoriasis is a chronic skin disease, characterized by the appearance of red, well defined plaques of various sizes, containing scales and located on different parts of the body, the most frequently affected regions being the knees, elbows, back and head. The cause of psoriasis is complex, involving genetic predisposing factors, autoimmune reactions, allergies of different natures, as well as a series of triggers: endocrine, neuropsychiatric, digestive, and metabolic disorders, viral infections, various foci of infection, local trauma, higher respiratory tract infections. Symptoms of psoriasis can vary from person to person, and the appearance of the characteristic eruptions, called psoriatic plaques, varies too. Generally, these are: red patches covered by thickened skin, portions of losing skin, portions of very dry chapped skin, which can bleed, pain, burning, itching, thickened chapped nails, in some cases swollen painful joints. Current treatments for psoriasis consist of: topically applied creams and ointments, phototherapy and/or systemic therapies consisting of oral or injectable medication. Topically applied treatments have the disadvantage that only work for mild forms of the disease, and only after a lengthy application, of about 6 weeks. Phototherapy has the disadvantage that involves using expensive devices for artificial ultra-violet light, which are found only in few specialized health care units and can be performed only under the supervision of a dermatologist. Systemic treatments, the only ones that work for severe forms of psoriasis, all have potentially severe side effects, especially in the kidneys. Furthermore, none of these known treatments definitively avoids the recurrence of psoriatic plaques on the skin.

The present invention discloses chlorine or bromine salts of cetylpyridinium for use in the treatment of porphyria and psoriasis, as well as methods of treatment of these diseases by using hydro-alcoholic solutions of cetylpyridinium chloride or bromide.

The inventor studied the effects of cetylpyridinium chloride and bromide application on some diseases and succeeded to achieve absolutely outstanding and unexpected results in the treatment of porphyria and psoriasis.

One aspect of the present invention relates to chlorine or bromine salts of cetylpyridinium for use in a method of treatment of the cutaneous or acute porphyrias. For this use, cetylpyridinium chloride or bromide is advantageously applied in the form of a hydro-alcoholic solution, preferably a 10% hydro-alcoholic solution. Said solution is applied topically, for example by spraying, to an area of the patient's skin unaffected by the disease, preferably the surface area of the lower limbs or an area of an equivalent size. The preferred method of treatment consists in applying a 10% hydro-alcoholic solution of cetylpyridinium chloride or bromide by spraying, once a day for 10 to 15 consecutive days. Preferably, said solution is applied during the first 4-5 days on the surface of both lower limbs or an equivalent surface, and during the next 5 to 10 days it is applied to the surface of one leg, alternately. In children, and in people with sensitive skin, the solution will be applied on one leg each day, alternately, for the duration of the treatment. Special attention will be given to areas with more delicate skin, such as the area from the back of the knee, where the skin can become sensitive after applying the solution, in which case the application will avoid these areas. In cases of irritation, that area should be avoided or the treatment should be discontinued for 1-2 days.

By using hydro-alcoholic solutions of cetylpyridinium chloride or bromide for a method of treating cutaneous or acute porphyrias aforementioned, skin lesions caused by cutaneous porphyria heal, skin fragility disappears, and sunlight exposure is no longer harmful so the patient can expose to sunlight without restrictions. The data obtained showed that complete healing occurs, since none of the patients thus treated showed any relapse.

According to the trial below, which was run in 2006 in a hospital, a 10% hydoalcoholic solution of cetylpyridinium chloride was given to a group of 7 patients suffering from cutaneous late porphyria, as shown in the table:

| | | | Total urinary | | Uroporphyrins | | Coproporphyrins | | Etanol |
|---|---|---|---|---|---|---|---|---|---|
| Nr. | | | porphyrin μg/24 h | | μg/24 h | | μg/24 h | | consump- |
| crt. | Name | Age | Found | Normal | Found | Normal | Found | Normal | tion |
| 1 | P. E. | 46 | 2005 | <220 | 970 | 15-50 | 1035 | 35-150 | — |
| 2 | G. N. | 52 | 292.6 | <220 | 70 | 15-50 | 222.5 | 35-150 | YES |
| 3 | P. A. | 53 | 229 | <220 | 110 | 15-50 | 129 | 35-150 | — |
| 4 | G. I. | 61 | 1049 | <220 | 674 | 15-50 | 375 | 35-150 | YES |
| 5 | B. N. | 65 | 306 | <220 | 154 | 15-50 | 121 | 35-150 | YES |
| 6 | D. I. | 57 | 425 | <220 | 180 | 15-50 | 210 | 35-150 | — |
| 7 | M. C. | 60 | 350 | <220 | 162 | 15-50 | 173 | 35-150 | — |

The 10% hydro-alcoholic solution of cetylpyridinium chloride was applied by spraying once a day for 10 days in a row on the lower limbs of the patients, then the solution was homogeneously spread over the entire surface of the lower limbs. After applying this method of treatment, the various skin lesions caused by porphyria in patients were cured. By the end of 2015, i.e. 10 years later, none of these patients, some of which having been physically handicapped for many years before administering the treatment, reported any recurrence of disease symptoms.

Another aspect of the present invention relates to chlorine or bromine salts of cetylpyridinium for use in a method of treatment of psoriasis. For this use, cetylpyridinium chloride or bromide is advantageously applied in the form of a hydro-alcoholic solution, preferably a 20% hydro-alcoholic solution. Said solution is administered topically, for example by spraying, to the surface of the skin affected by psoriasis. The preferred method of treatment consists of one or more cycles of treatment. A cycle of treatment comprises applying the 20% hydro-alcoholic solution of the cetylpyridinium chloride or bromide by spraying it onto the affected area, three times separated by intervals of about 10 minutes, which time is necessary for the solution to be absorbed and for the skin area of application to be dried, followed by applying to the affected area of an ointment containing 5-20% ichthyole as an active ingredient and then covering the affected area with an occlusive dressing for 3 days. The treatment cycle is repeated, as needed, till curing, preferably for 3 or 4 times, i.e. 9-12 days. The use of an ointment containing ichthyole as an active ingredient in a concentration of 5% to 20% for the treatment of psoriasis is known. For an use in a preferred method of treatment, the ointment containing ichthyole as an active ingredient is an ointment comprising, in percent by weight: 0.003% flumethasone pivalate, 0.1% triamcinolone acetonide, 35% lanolin, 10% liquid paraffin, 45% petrolatum and the remainder up to 100% ichthyole. Optionally, the composition may further comprise 4% tar by weight.

This method of treatment is effective for all forms of psoriasis, from the mild to the severe forms, has no side effects, involve low costs, it is administered for a much shorter time than known topical treatments and without discomfort of patients as compared to injectable treatments, to treatments performed by specialists in specialized institutions or to those lasting up to 6 weeks.

In case of new recurrences of the disease in the form of small red spots, the 20% hydro-alcoholic solution of cetylpyridinium chloride or bromide will be applied thereon. In this case, the solution according to the invention is administered topically, locally, for example from a dropper vial or a minispray, for 2 to 5 times daily at intervals of 5-10 minutes, for a period of 3-4 consecutive days. Following this treatment, the red spots recede (disappear) without plaque developing, and the affected area heals quickly and very cost-effective. This allows us to control psoriasis in the treated areas.

1. (canceled)

2. Chlorine or bromine salts of cetylpyridinium for use in the treatment of porphyrias wherein the chlorine or bromine salts of cetylpyridinium are used for topical administration to human patients suffering from porphyria, in the form of a 10% hydro-alcoholic solution of cetylpyridinium chloride or bromide, by daily spraying, once a day, on a skin area of the patient unaffected by the disease, preferably on the surface of both lower limbs or on a skin area of an equivalent size, for 10 consecutive days.

3. Chlorine or bromine salts of cetylpyridinium for use in the treatment of psoriasis according to claim 2, wherein the chlorine or bromine salts of cetylpyridinium are used for topical administration to human patients suffering from psoriasis, in the form of a 20% hydro-alcoholic solution of cetylpyridinium chloride or bromide, by administering 3-4 consecutive cycles of treatment, each cycle comprising spraying the 20% hydro-alcoholic solution of cetylpyridinium chloride or bromide on the affected area three times at intervals of 10 minutes, followed by the administration of an ointment comprising 5 to 20% ichthyole and by covering the treated area with an occlusive dressing which is kept for 3 days.

4. Chlorine or bromine salts of cetylpyridinium for use in the treatment of psoriasis according to claim 3, wherein said ointment comprising ichthyole, comprises in percent by weight: 0.003% flumethasone pivalate, 0.1% triamcinolone acetonide, 35% lanolin, 10% liquid paraffin, 45% petrolatum, optionally 4% tar and the remainder up to 100% ichthyole.

5. Chlorine or bromine salts of cetylpyridinium for use in the treatment of psoriasis according to claim 2, wherein the chlorine and bromine salts of cetylpyridinium are used for the treatment of a new relapse of psoriasis in the form of small red spots, by topical administration, to the affected areas of human patients suffering from a relapse of psoriasis, of a 20% hydro-alcoholic solution of cetylpyridinium chloride or bromide, daily for 2 to 5 times at intervals of 5-10 minutes, for a period of 3-4 consecutive days.
